(19) United States
(12) Patent Application Publication
(10) Pub. No.: US 2017/0050048 A1
(43) Pub. Date: Feb. 23, 2017

(54) METHOD OR APPARATUS FOR WRINKLE TREATMENT

(71) Applicant: Oren Aharon, Haifa (IL)

(72) Inventor: Oren Aharon, Haifa (IL)

(21) Appl. No.: 15/220,405

(22) Filed: Jul. 27, 2016

(30) Foreign Application Priority Data
Aug. 10, 2015 (IL) 240487

(51) Int. Cl. A61N 5/06

(52) U.S. Cl. CPC A61N 5/0616; A61N 2005/0626; A61N 2005/067

(57) ABSTRACT

A method and apparatus are disclosed for skin wrinkle removal. After applying a tube encircling a relatively small skin area along the wrinkled line, a set of laser beams will be activated. The laser beams are mounted around the tube, pointing to its center to create a mutual point of irradiation located just below the skin's wrinkle line. Optical energy from lasers is directed to a point under the skin by radiating through the transparent tube. For ease of use, the encircled skin portion is lifted, preferably by vacuum, enabling laser radiation to penetrate under the skin from outside the lifting tube. The apparatus is suitable for treating skin wrinkles by targeting the sub skin melanin without damaging skin surface. The apparatus may also be used for other aesthetic face treatments, including disorders located around the eye. The used laser radiation is in the wavelength region of 1500 to 1800 nm, which is regarded to be eye safe. Other laser wavelength could be used, especially in the 700 nm region with confidence, since most radiation is enclosed within the skin portion on the transparent tube. The treatment energy density level on the skin penetration point is relatively low and thus not harmful to the epidermis. However, heating effect is maximized at the central point just beneath the wrinkle—which causes damage to the collagen. Later on, when healing occurs, the skin will be stretched and wrinkles will be diminished. This technology is less damaging to the skin than that of the prior and targets only the under wrinkle collagen. Statistically, most of the recent clinical trials showed a histological evidence of new collagen formation and significant improvement in facial rhytides. Direct, under the skin irradiation, as in our art, will be less damaging to the skin epidermis yielding improved overall results.

### BACKGROUND OF THE INVENTION

1. Field of the Invention

The present invention is related to the field of light-based skin treatments. More specifically, the invention is related to the utilization of light sources for wrinkle treatment. Other relevant disorders could be similarly treated by irradiating the point of interest located beneath the skin level. Radiation is applied to a lifted skin portion from its periphery through a transparent tube, causing the temperature at the center point under the skin to peak. By irradiating from multiple points around a lifted part of the skin, damage to the skin itself is relatively small, since it is applied from different points—each radiating with low level laser energy. By lifting a relatively small skin portion, the epidermis and dermis are stretched and thinned enabling better penetration to the subcutis.

2. Description of the Related Art

The subcutis is the innermost layer of the skin, and consists of a network of fat and collagen cells. The blood vessels, nerves, lymph vessels, and hair follicles also cross through this layer. The thickness of the subcutis layer varies throughout the body and from person to person. Whereby light is absorbed or transmitted by skin layer surrounding the center and the created temperature and radiation peak is maximal at the point of interest usually located beneath the wrinkle disorder. For treatment application, the skin is lifted into a transparent tube enclosing an area around the wrinkle line and laser energy is applied from its perimeter through the transparent material. The device is preferably offered as an over the counter product.

In their article, published by Lasers Med Sci (2007) 22: 1-3 DOI 10.1007/s10103-006-0406-x (authors: Rachel Lubart, Harry Friedmann, Ronit Lavie, Leonardo Longo, Julia Jacobi, Ohad Baruchin and Abraham M. Baruchin) titled “A reasonable mechanism for visible light-induced skin rejuvenation”, the authors speculate that when high concentrations of visible light ROS (reactive oxygen species) are formed, they destroy old collagen fibers, encouraging the formation of new ones. Moreover, when at inner depths of the tissue, low amounts of ROS are formed; they stimulate fibroblasts and collagen metabolism, resulting in an improvement of skin texture.

For light-colored skin, penetration through the skin is preferable with a wavelength of around 670 nm. This wavelength region also provides minimal absorption by Oxyhemoglobin combined with minimum water absorption.

Moreover, scientists have observed that exposure of the skin to yellow, orange and/or red light leads to higher production of collagen, due to the fact that collagen and elastin are stimulated around this color range, thus improving the elasticity of the skin.

The effects of aging on the dermal skin layer are significant in causing wrinkles and local sagging. The dermal layer thin, less collagen is produced, and the elastin fibers that provide elasticity wear out. These changes in the scaffolding of the skin cause the skin to wrinkle and sag. By applying laser radiation to the skin scaffolding, new collagen growth is stimulated by the body's natural healing process, thus by selectively targeting the under skin collagen a healthy new scaffold is produced for smoother dermis surface. The field of the invention is a method of inducing limited damage by laser radiating to the under skin scaffold, creating a self healing process. Unlike prior art devices intended for wrinkles removal, which apply high levels of radiation to the epidermis causing skin damage, the proposed art applies low laser dosage creating a focal point under the epidermis directly targeting the collagen.

The popularity of laser treatment of skin and dermatological related disorders is usually applied in the controlled environment of a medical facility. Prior art face tissue tightening is achieved with a very high intensity laser or IPL source applied to the upper surface of the skin.

Laser radiation penetrates through the epidermis into the skin collagen and burns some of the collagen cells. By doing so, the natural healing process of body starts to work and stimulates new collagen production in the skin.

In order to penetrate and achieve high fluency at the subcutis skin layer, the needed amount of radiation at the skin level is very high, preventing such technologies to be available as over the counter devices.

Typically, irradiation technique is applied to the skin surface from above, even if the target lies beneath the skin level—this may have some adverse results to the upper skin level due to the high levels of fluence needed in order to achieve deep penetration. Applying the light to the skin from above has some limitations such as a limited maximum power level and uncontrolled laser propagation through the skin due to scattering. Those effects are major obstacles for wrinkle treatment especially in the vicinity of the users' eyes.

The disclosed art prevents damage to the epidermis by applying the laser radiation through a relative large circumference area in a perimeter of a lifted skin section that will be confined by an external tube.

Prior art systems are not intended to maximize the under the skin laser fluence or temperature by irradiating through a tubular transparent device. Moreover, these prior art systems are not intended to block under-the-skin laser propagation; making the treatment potentially harmful if it reaches the eye vicinity.

Applying a vacuum to the skin for massaging purposes is a known prior art procedure for treatment of cellulites. As the vacuum is being applied, a skin fold is formed, causing the underlying cellulites format to be raised above the surrounding surface, while the movement of the suction device performs the massage.

The disclosed art may use a miniature tubular suction device for raising a small skin area of a few millimeters, creating a raised section with a clear edge perimeter. Light radiation is applied through a transparent tube or orifice directly under the raised skin, maximizing the temperature at the center of the wrinkled dermis. The target layer is preferably the subcutis. The applied vacuum has no therapeutic effect and it is used as a mechanical lifting device of a small skin portion.

The subcutis is the innermost layer of the skin and consists of a network of fat and collagen cells. It extends below the dermis with varying depth, depending on the anatomic location.

Collagen is the most abundant protein in the skin and is the major component of the extracellular matrix. Collagen makes up 70-80% of the dry weight of the skin and gives the dermis its mechanical and structural integrity.

A need therefore exists for treating the subcutis below the skin level, without damaging the epidermis, to remove unwanted wrinkles by applying the radiation to a perimeter of a raised portion of skin. The actual applied dosage at the perimeter is relatively low, but the converging sub skin radiation at its center is high, enough to apply the required dosage directly under the wrinkled area.

It is an object of the present invention to provide a method and apparatus for the treatment of wrinkles and other under-skin disorders by a non-ablative laser or light system applied to the perimeter of a raised portion of skin, at wavelengths shorter than 1600 nm without damaging the surface of the skin or the epidermis.

It is an additional object of the present invention to provide a method and apparatus by which the laser energy is contained by the tube raising the skin to prevent laser radiation to travel through the skin to unwanted regions

It is an additional object of the present invention to provide a method and apparatus by which the number of required treatments for fine wrinkles removal is reduced in comparison to that of the prior art.

It is a further object of the present invention to provide an apparatus for vacuum-skin lifting device to assist applying laser and light-based under the skin treatment.

Other objects and advantages of the invention will become apparent as the description proceeds.

According to current absorption curves, lasers operating at the spectral range of approximately 650 nm to 750 that are not very well absorbed by blood vessels and Oxyhemoglobin, are being absorbed by melanin. Moreover, water absorption is minimal allowing utilization for wrinkle smoothing using our method. The energy density at skin penetration will be much lower than required by other techniques, while maximizing level density at the center just below the wrinkle at the rich melanin layer.

Treatments of wrinkles with other wavelengths are also possible due to a different phenomenon of water absorptions at the range of IR starting from 1000 nm to 1700 nm. This will be manifested by temperature raise at the perimeter with peak at its center.

The aforementioned prior arts efforts to minimize blood vessels damages, by cooling the upper skin level, helps in some cases to avoid unnecessary damage to skin structures, are difficult and totally unnecessary in the presented art.

### SUMMARY OF THE INVENTION

The present invention is directed to apparatus for low power laser sub skin treatments. The apparatus comprises a skin lifting device which is transparent to laser or other radiation. A vacuum or a mechanical lifting mechanism is applied to said transparent lifting device or tube, whereby a small usually circular skin portion is drawn to said vacuum tube. Then laser radiation is applied from the tubes' perimeter along its radii. Preferably, the tube's center coincides with a skin disorder such as a wrinkle, and the applied radiation converges at tube center just below the wrinkle. The efficacy and utility of the apparatus are achieved by the fact that maximum heat and radiation are just below the wrinkle to be smoothened, and since the laser dosage at entry point is low the damage to the skin is minimized.

In an embodiment of the invention, an apparatus for cosmetically removing skin wrinkles by laser irradiation applied from several directions to a lifted enclosed skin area comprises:
A tube for lifting and enclosing an area of around the wrinkle.
Multiple lasers irradiating through the perimeter of the tube and into the center located below the lifted skin area.
A vacuum pump to evacuate the air from transparent tube lifting a skin portion.
Means for applying a vacuum to said vacuum tube.
A controller for driving the laser according to pre selected time table.
An on/off activating device.
An apparatus according to the above description wherein said skin lifting is performed by creating vacuum in the transparent tube.
An apparatus according to the above description wherein said skin lifting is performed by creating vacuum in an opaque tube with orifices for laser beams.
An apparatus according to the above description where the laser sources comprises a laser of wavelength of 1500 to 1800 nm
An apparatus according to above descriptions wherein said source comprises a laser of 670 to 720 nm.
An apparatus according to above descriptions wherein the laser sources have both wavelength 670 to 720 nm and 1400 to 2000 nm
An apparatus according to above descriptions where the transparent tube is coated with a mirror reflective coating with uncoated area at the laser input aperture.

To summarize:
The wavelength of the light ranges from 400 nm to 1800 nm with preferred bands of 670 nm to 720 nm and 1400 to 200 nm.
The level of applied vacuum within the vacuum chamber ranges from approximately 0 to approximately 1 atmosphere;
The transmitting element is transmitting the light in a direction located under the lifted skin portion along the said tube radii.
The vacuum tube is provided with a rim for sealing the peripheral contact area between the skin surface and its circular end.

### BRIEF DESCRIPTION OF THE DRAWINGS

FIG. 1 is a schematic graph which illustrates the propagation of light radiation from a radiating hand piece located above the skin, versus the skin layers absorption.

FIG. 2A is a schematic drawing which illustrates the absorption ratio as a function of wavelength giving values and percentage to a specific wavelength.

FIG. 2B shows the penetration depth as a function of wavelength.

FIG. 3 is a schematic drawing of the new ad device.

FIG. 4 is a cross-section of the raised skin formation, showing the thinning of the upper layer.

FIG. 5 is a cross-section of the apparatus, showing the multiple laser light beams irradiation creating a converging area under the wrinkle.

### DETAILED DESCRIPTION OF EMBODIMENTS

The present invention is directed towards an apparatus which is provided with a unit for lifting a preferably round shape of skin. The skin is lifted prior to the firing of multiple lasers around the raised portion. The air suction unit comprises a miniature vacuum pump and a hose through which air is evacuated from a tubular device applied to the skin and intense light passes through its wall directly to the elevated skin level. During the operation of the vacuum pump, the vacuum level within the vacuum chamber is increased and a special sensor checks vacuum level before enabling laser firing option.

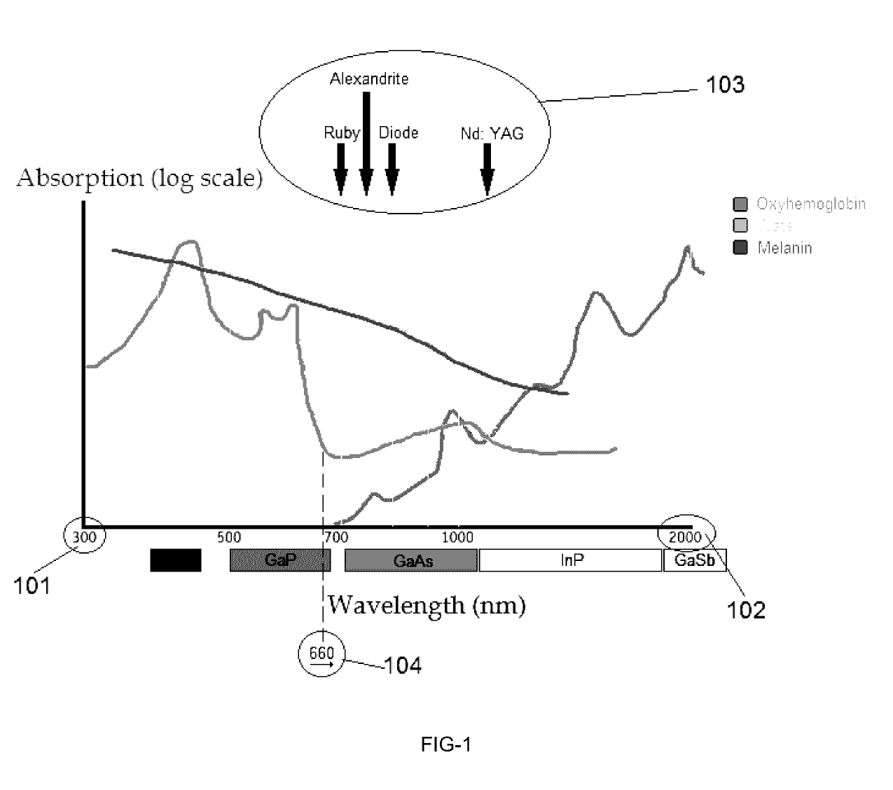

FIG. 1 illustrates the propagation of an intense pulsed laser or light beam, the wavelength of which is in the visible or near infrared region of the spectrum, i.e. shorter than 2000 nm, and longer than 300 nm and denoted as 101 and 102 respectively, popular lasers are denoted as 103 for minimizing damage to Oxyhemoglobin. A wavelength longer than 660 is a good choice and denoted as 104.

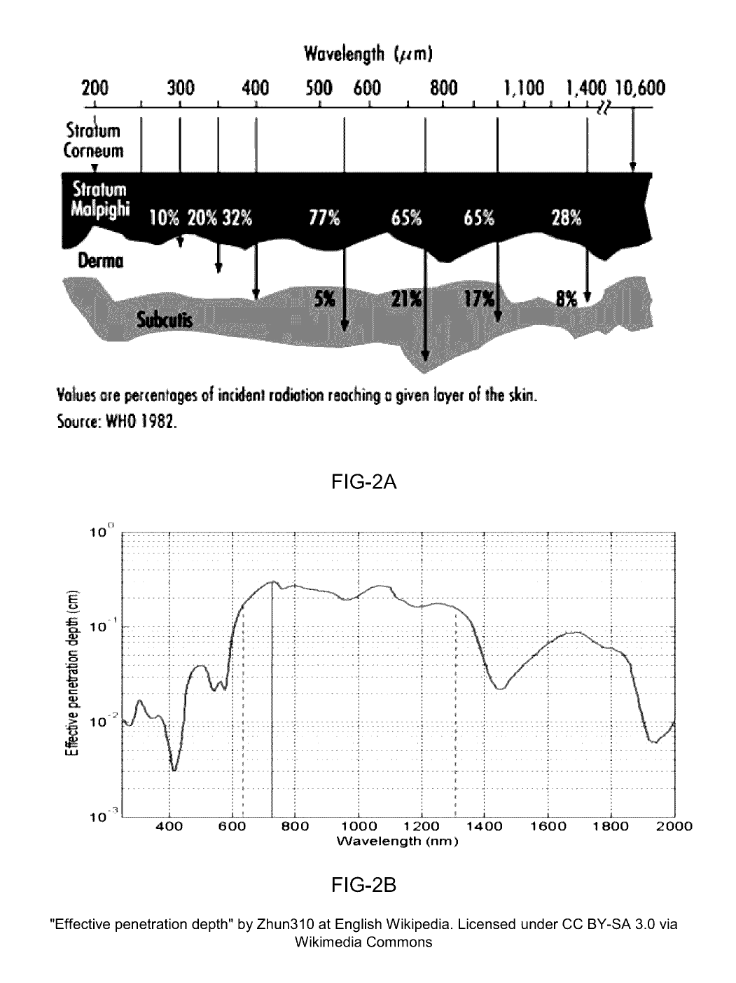

FIG. 2A shows the laser power penetration to the subcutis as a function of wavelength, showing that most of the laser energy is absorbed by the skin's upper level, thus limiting the maximum power level that can be delivered through radiation from above. FIG. 2B shows penetration depth in cm as a function of wavelength.

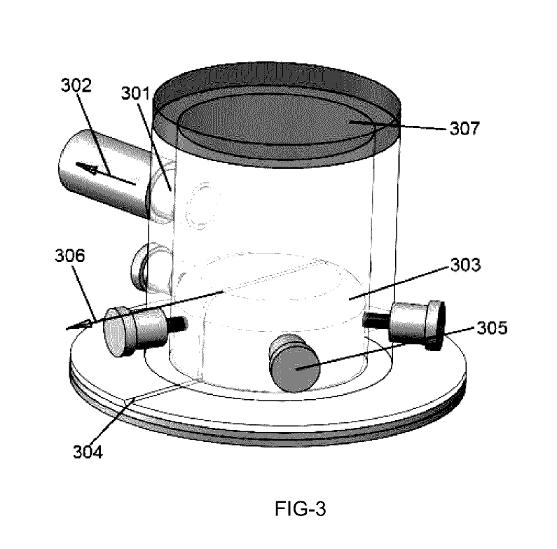

FIG. 3 illustrates a preferred configuration, where vacuum application is performed through an orifice denoted as 301, and air flow direction is denoted as 302. The application of the vacuum draws the skin, creating a convex surface 303, a schematically drawn wrinkle 304, is temporary lifted, creating a formation of skin fold which is raised in respect to the adjoining skin. The raised formation is irradiated by multiple lasers 305 on its perimeter to create significant fluencies directly under the raised skin. By moving the device along the wrinkle direction 306 and applying laser pulses along movement—the wrinkled line is treated. Due to the elasticity of skin, skin fold returns to its original configuration, upon subsequent movement of the device, while another skin fold is formed and treated. A transparent window 307 allows user to align the device to the wrinkled area to be treated.

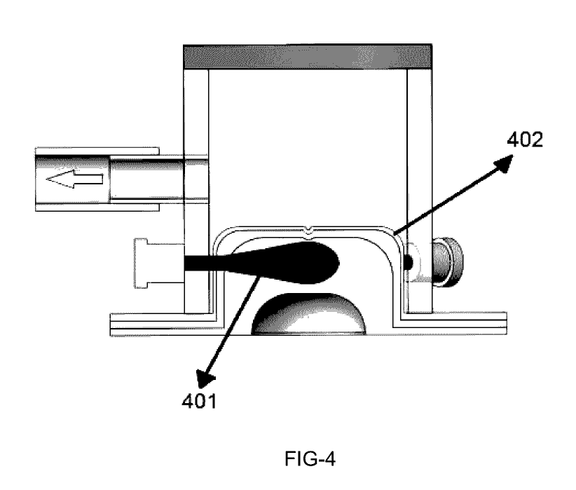

FIG. 4 illustrates a section perpendicular to wrinkle direction, showing the laser beam penetration and the skin thinning effect improving the laser beam transmittance and delivery to subcutis area. 401 shows the skin thinning within the tube, as compared in respect to the adjoining skin, 402 shows typical laser radiation from one of the lasers disposed at the perimeter of the raised skin.

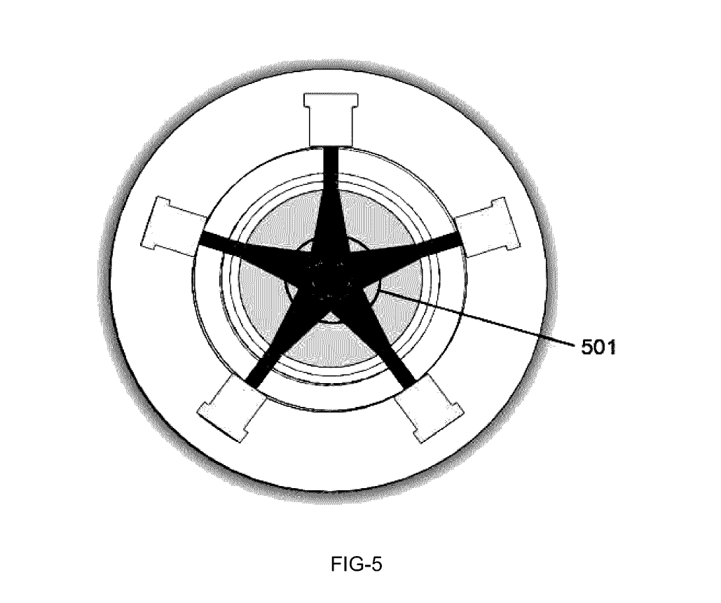

FIG. 5 illustrates a cross-section of the apparatus according to an embodiment of the invention, which is disclosed in FIG. 3, showing the multiple laser light beams irradiation under the skin path creating a converging area denoted as 501 and located under the wrinkle.

Although the application of a vacuum to skin surface has been employed in prior art, many significant differences between prior art apparatus and the presented device are obvious;
The prior art application of vacuum is intended to remove smoke or vapors caused by the light-based ablation of a skin surface. The present invention, in contrast, uses vacuum for raising the skin to allow the multiple directed beams of optical energy to interact under the skin surface, and are targeted to subcutis skin structures.
The present invention employs a vacuum chamber overlying a treated area not for performing any treatment, but rather to be used as an auxiliary means for skin lifting allowing the treatment lasers to perform. Other skin lifting techniques could be used, however vacuum skin lifting is preferable.

Apparatus for under skin light treatment will preferably follow some stages:
Once the vacuum chamber has been placed on the skin surface a suitable skin contact will activate pump.
The vacuum applying mode is equipped with a sensor (not shown) enabling laser firing only if proper vacuum level is reached.
Once the vacuum level within the vacuum chamber has reached a certain level and consequently the skin is raised- laser can be activated manually or automatically while the device is moving along the wrinkle.
Following termination of the treatment pulses at a certain area, the device can be further moved to a different area.
Treatment is periodically repeated about once per week for several weeks.

What is claimed is:

1. An apparatus for removing skin wrinkles comprises:
A transparent tubular suction device for lifting and enclosing a part of the skin around the wrinkle.
Multiple lasers irradiating through the tube's perimeter to a center point located below the lifted skin area.
A controller for driving the laser according to pre-selected time table.
An on/off activating device.

2. An apparatus according to claim 1, wherein said skin lifting is performed by creating a vacuum in the transparent tube.

3. An apparatus according to claim 1, wherein the tube is opaque and has openings for laser radiation.

4. An apparatus according to claim 1 where the laser sources comprises lasers having a wavelength of 1500 to 1800 nm.

5. An apparatus according to claims 3 and 4 wherein said source comprises of lasers having wavelength between 670 to 720 nm.

6. An apparatus according to claims 3 and 4 wherein the laser sources have both wavelengths 670 to 720 nm and 1400 to 2000 nm.

7. An apparatus according to claims 1 to 6 wherein the transparent tube is coated with a mirror reflective coating with uncoated area at the laser input aperture.
